(19) United States
(12) Patent Application Publication
(10) Pub. No.: US 2011/0201013 A1
(43) Pub. Date: Aug. 18, 2011

(54) Assays and Devices For Identifying Pathogens

(76) Inventor: Norman J. Moore, North Berwick, ME (US)

(21) Appl. No.: 12/446,115

(22) PCT Filed: Oct. 22, 2007
(86) PCT No.: PCT/US07/82096

§ 371 Date: Nov. 9, 2010

Related U.S. Application Data
(60) Provisional application No. 60/853,264, filed on Oct. 20, 2006.

(51) Int. Cl. C12Q 1/68

(52) U.S. Cl. 435/6.19

(57) ABSTRACT

Provided are methods and devices for identifying pathogens based on protein translation from ribosomes isolated from the pathogens.

### BACKGROUND OF THE INVENTION

Detection of the bacteria that have infected a subject, including metabolites, nucleic acids, and proteins thereof, is a fundamental component in the diagnosis and treatment of medical disorders, as well as in research. A number of methodologies are currently in use for detection. These methodologies can generally be divided into antibody-based diagnostic assays for proteins, either components of the bacteria or byproducts of the disease, and diagnostic assays for nucleic acids, such as the genetic material encoding a component of the bacteria.

Existing methodologies for nucleic acid detection require a high degree of technical competence for reliability due to the complexity of the reaction conditions (for example, PCR requires thermocycling) and may be extremely sensitive to contamination resulting in false positives; they are difficult to use quantitatively rather than qualitatively and thus their sensitivity is compromised. Further, they often take hours to complete. Methodologies for protein detection generally rely on conjugation of an enzyme, usually to additional components of the assay, to increase signal generation and amplification. The use of these additional ligands increases the noise of the system, with higher background and false positives, and necessitates several levels of control reactions.

### SUMMARY OF THE INVENTION

Provided are methods of using ribosomes from pathogens to identify the pathogens, e.g., for diagnostic or treatment purposes. Ribosomes are specific to a particular pathogen, and thus the identity or presence of a pathogen in a sample may be determined based on determining whether a polypeptide is able to be produced using a nucleic acid template with ribosomes isolated using a binding agent specific for the pathogen's ribosomes. The methods may be performed at a single temperature, avoiding complex reaction conditions. Further, the amplification of the template by the ribosomes is rapid—at least about 20,000 target molecules are produced every 10 seconds—and is very sensitive. Still further, the amplification reaction itself is generic, with only the nucleic acid template and binding agent used to immobilize the ribosomes differing from test to test based on the pathogen to be detected. Thus, manufacturing of test kits and devices for the practice of the methods could be drastically simplified.

The methods may be incorporated into any test format or device suitable for the practice of the methods. Also provided are kits, reagents, etc. for the practice of the methods.

Further objectives and advantages of the present invention will become apparent as the description proceeds. To gain a full appreciation of the scope of the present invention, it will be further recognized that various aspects of the present invention can be combined to make desirable embodiments of the invention.

### BRIEF DESCRIPTION OF THE DRAWINGS

FIG. 1 depicts a schematic of an exemplary embodiment of the use of ribosomal amplification to detect Streptococcus A bacteria.

### DETAILED DESCRIPTION OF THE INVENTION

Unless defined otherwise above, all technical and scientific terms used herein have the same meaning as commonly understood by one of ordinary skill in the art to which this invention belongs. Where a term is provided in the singular, the inventor also contemplates the plural of that term. The nomenclature used herein and the procedures described below are those well known and commonly employed in the art.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. The names of the natural amino acids are abbreviated herein in accordance with the recommendations of IUPAC-IUB.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. In exemplary embodiments, antibodies used with the methods and compositions described herein are derivatives of the IgG class.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The terms “comprise” and “comprising” is used in the inclusive, open sense, meaning that additional elements may be included.

The term “including” is used herein to mean “including but not limited to”. “Including” and “including but not limited to” are used interchangeably.

The term “mRNA” refers to messenger RNA, or the RNA that serves as a template for protein synthesis in a cell. The sequence of a strand of mRNA is based on the sequence of a complementary strand of DNA comprising a sequence coding for the protein to be synthesized.

“Nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The term “pathogen” refers to any organism which may cause disease in a subject, such as a bacterium, fungus, parasite, virus, etc.

“Protein” (if single-chain), “polypeptide” and “peptide” are used interchangeably herein when referring to a gene product, e.g., as may be encoded by a coding sequence. When referring to “polypeptide” herein, a person of skill in the art will recognize that a protein can be used instead, unless the context clearly indicates otherwise. A “protein” may also refer to an association of one or more polypeptides.

The term “sample” refers to any sample potentially containing pathogens containing ribosomes.

The term “ribosomal RNA” or “rRNA” refers to the RNA component of ribosome subunits. Ribosomes and their subunits are described further below.

Provided in one aspect is a method, comprising:
(a) contacting a support comprising a binding agent capable of binding a ribosome with a sample comprising ribosomes;
(b) washing the support after contact with the sample;
(c) incubating the support in the presence of a solution comprising mRNA and amino acids under conditions suitable to allow ribosomes bound to the binding agent to translate the mRNA in the solution into a polypeptide; and
(d) determining the presence of the polypeptide.

In certain embodiments, the ribosomes are specific to a pathogen and the method further comprises determining the presence of the pathogen in the sample based on the presence of the polypeptide.

Provided in another aspect is a method, comprising:
(a) capturing ribosomes on a support using a binding agent capable of binding the ribosomes;
(b) incubating the captured ribosomes with a solution comprising mRNA and amino acids; and
(c) detecting the polypeptide produced by the incubated, captured ribosomes.

Ribosomes are ribonucleoproteins which are present in both prokaryotes and eukaryotes. They comprise about two-thirds RNA and one-third protein. Ribosomes are the cellular organelles responsible for protein synthesis. During gene expression, ribosomes translate the genetic information encoded in a messenger RNA into protein (Garrett et al. (2000) “The Ribosome: Structure, Function, Antibiotics and Cellular Interactions,” American Society for Microbiology, Washington, D.C.).

Ribosomes comprise two nonequivalent ribonucleoprotein subunits. The larger subunit (also known as the “large ribosomal subunit”) is about twice the size of the smaller subunit (also known as the “small ribosomal subunit”). The small ribosomal subunit binds messenger RNA (mRNA) and mediates the interactions between mRNA and transfer RNA (tRNA) anticodons on which the fidelity of translation depends. The large ribosomal subunit catalyzes peptide bond formation—the peptidyl-transferase reaction of protein synthesis—and includes (at least) two different tRNA binding sites: the A-site which accommodates the incoming aminoacyl-tRNA, which is to contribute its amino acid to the growing peptide chain, and the P-site which accommodates the peptidyl-tRNA complex, i.e., the tRNA linked to all the amino acids that have so far been added to the peptide chain. The large ribosomal subunit also includes one or more binding sites for G-protein factors that assist in the initiation, elongation, and termination phases of protein synthesis. The large and small ribosomal subunits behave independently during the initiation phase of protein synthesis; however, they assemble into complete ribosomes when elongation is about to begin.

Accordingly, as used herein, the term “ribosome” refers to a complex comprising a large ribosomal subunit and small ribosomal subunit. The large ribosomal subunit and small ribosomal subunit are 50 S and 30 S subunits respectively in bacteria and 60 S and 40 S subunits respectively in eukaryotes.

Protocols describing the preparation of samples comprising ribosomes are available in the literature and can be adapted where needed by those skilled in the art. For example, the preparation of ribosomes from bacteria can be done essentially as described by Youmans and Youmans, 1965 and adapted as described by Gregory et al., 1983. In general, but particularly when using virulent Microbes (pathogenic), is recommended to kill the cells prior to further use, for example by treatment with formalin as described by Michalek and McGhee, 1977, and adjust concentrations to 10⁸ bacterial or fungal cells/ml or 10⁷ protozoa/ml. The preparation can be established to be sterile when no multiplication occurs upon inoculation on Sheep blood and Mitis Salivarius agars (DIFCO) or other adapted rich culture medium. Aliquots are stored at −80.degree. C. Subsequently they are thawed rapidly at 37.degrees. C., and 1 g of whole cells is re-suspended with 1 g of micro-glass beads (0.17-0.18 mm) in 1 ml of PMB to which 3 .mu.g/ml Dnase (SIGMA) is added. The cells are disrupted by shaking for three 2-minute cycles in a Braun homogenizer. Intact cells and debris are removed by two centrifugations (27.000.times.g followed by 47.000.times.g; 10 minutes each).

Preparation of ribosomes from fungi and protozoa follow essentially the same procedure but require adaptation of culture conditions and lysis methods. Given that culture conditions of cultivatable pathogenic microbes are widely available in published literature, preparation of ribosomes from such microbes is well within the possibilities of a person skilled in the art.

Integrity of the ribosomal subunits is important. In particular the stabilization of enclosed large ribosomal RNA's by divalent cations such as provided by MgCl₂, concentration which may need adaptation depending on the microbe and extraction protocol methods used. The ribosomes in the supernatant can be harvested by centrifugation at 180.000 to 250.000.times.g for 2 to 3 hr and then subjected to 5 successive washes in PMB at 180.000 to 250.000.times.g for 2 to 3 hr each. The ribosomal preparation is then clarified twice by two 20-min centrifugations at 47.000.times.g and the supernatant is filtered through a sterile 0.45 micron Millipore filter (Millipore Filter Corp.). Non-dissociated (=intact) ribosomes can be prepared from gram-negative, Rnase-minus mutant bacteria such as Escherichia coli MRE600 following the method of Staehilin et. al., 1969, with modifications as described by M. M. Yusupov and A. S. Spirin. 1988. The preparations can then adjusted to, for example, 20 mg/ml on the basis of protein content by standard protein quantification methods, using, for example, bovine serum albumin as a standard, and maintained at −80.degree. C. until used. Characterization of the ribosomal fraction and purity can be determined by spectral analysis at 235, 280 and 260 nm in order to determine the contamination of ribosomal RNA by DNA polyacrylamide gel electrophoresis permits to evaluate the presence of ribosomal proteins and potential contaminating proteins. The degree of intactness can be evaluated by loading a sample of the original homogenate onto a 10% to 40% sucrose gradient, containing an appropriate concentration of Mg Cl₂ and centrifugation. The elusion profile of the sucrose gradient will show the different fractions: 100S=dimers of 70S ribosomes, 70S=intact ribosomes, 60S=interacting 50S and 30S ribosomal subunits, 50S=large ribosomal subunit, 30S=small ribosomal subunit, material less than 30S=degradation products and contaminants. In good preparations that target non-dissociated ribosomes, the 70S peak contains over 80% of all material. Optionally, the 70S peak containing the target non-dissociated ribosomes may constitute at least 50%, 60%, 70% or 90% of all material.

Pathogens that may be detected using the above methods include any organism comprising ribosomes. Organisms from which ribosomes may be isolated include, but are not limited to, the following:

Ribosomes from bacteria such as: Acinetobacter calcoaceticus, A. haemolyticus, Aeromonas hydrophilia, Bacteroides fragilis, B. distasonis, Bacteroides 3452A homology group, B. vulgatus, B. ovalus, B. thetaiotaomicron, B. uniformis, B. eggerthii, B. splanchnicus, Branhamella catarrhalis, Campylobacterfetus, C. jejuni, C. coli, Citrobacterfreundii, Clostridium difficile, C. diphtheriae, C. ulcerans, C. accolens, C. afermentans, C. amycolatum, C. argentorense, C. auris, C. bovis, C. confusum, C. coyleae, C. durum, C. falsenii, C. glucuronolyticum, C. imitans, C. jeikeium, C. kutscheri, C. kroppenstedtii, C. lipophilum, C. macginleyi, C. matruchoti, C. mucifaciens, C. pilosum, C. propinquum, C. renale, C. riegelii, C. sanguinis, C. singulare, C. striatum, C. sundsvallense, C. thomssenii, C. urealyticum, C. xerosis, Enterobacter cloacae, E. aerogenes, Enterococcus avium, E. casseliflavus, E. cecorum, E. dispar, E. durans, E. faecalis, E. faecium, E. flavescens, E. gallinarum, E. hirae, E. malodoratus, E. mundtii, E. pseudoavium, E. raffinosus, E. solitarius, Francisella tularensis, Gardnerella vaginalis, Helicobacter pylori, Kingella dentrificans, K. kingae, K. oralis, Klebsiella pneumoniae, K oxytoca, Moraxella catarrhalis, M atlantae, M. lacunata, M. nonliquefaciens, M. osloensis, M. phenylpyruvica, Morganella morganii, Parachlamydia acanthamoebae, Pasteurella multocida, P. haemolytica, Proteus mirabilis, Proteus vulgaris, Providencia alcalifaciens, P. rettgeri, P. stuartii, Serratia marcescens, Simkania negevensis, Streptococcus pneumoniae, S. agalactiae, S. pyogenes, Treponema pallidum, Vibrio cholerae, and V. parahaemolyticus.

Ribosomes from facultative intracellular bacteria such as: Bordetella pertussis, B. parapertussis, B. bronchiseptica, Burkholderia cepacia, Escherichia coli, Haemophilus actinomycetemcomitans, H. aegyptius, H. aphrophilus, H. ducreyi, H. felis, H. haemoglobinophilus, H. haemolyticus, H. influenzae, H. paragallinarum, H. parahaemolyticus, H. parainfluenzae, H. paraphrohaemolyticus, H. paraphrophilus, H. parasuis, H. piscium, H. segnis, H. somnus, H. vaginalis, Legionella adelaidensis, L. anisa, L. beliardensis, L. birminghamensis, L. bozemanii, L. brunensis, L. cherrii, L. cincinnatiensis, Legionella drozanskii L. dumoffli, L. erythra, L. fairfieldensis, L. fallonii, L. feeleii, L. geestiana, L. gormanii, L. gratiana, L. gresilensis, L. hackeliae, L. israelensis, L. jordanis, L. lansingensis, Legionella londiniensis L. longbeachae, Legionella lytica L. maceachernii, L. micdadei, L. moravica, L. nautarum, L. oakridgensis, L. parisiensis, L. pittsburghensis, L. pneumophila, L. quateirensis, L. quinlivanii, L. rowbothamii, L. rubrilucens, L. sainthelensi, L. santicrucis, L. shakespearei, L. spiritensis, L. steigerwaltii, L. taurinensis, L. tucsonensis, L. wadsworthii, L. waltersii, L. worsleiensis, Listeria denitrificans, L. grayi, L. innocua, L. ivanovii, L. monocytogenes, L. seeligeri, L. welshimeri, Mycobacterium abscessus, M africanum, M. agri, M. aichiense, M. alvei, M. asiaticum, g aurum, M austroafricanum, M. avium, M. bohemicum, M bovis, M. branderi, M. brumae, M. celatum, M. chelonae, M. chitae, M. chlorophenolicum, M chubuense, M. confluentis, M. conspicuum, M. cookii, M. diernhoferi, M. doricum, M duvalii, M. elephantis, M. fallax, M. farcinogenes, M. flavescens, M. fortuitum, M frederiksbergense, M. gadium, M. gastri, M. genavense, M gilvum, M. goodii, M. gordonae, M haemophilum, M. hassiacum, M. heckeshornense, M. heidelbergense, M hiberniae, M immunogenum, M. intracellulare, M. interjectum, M. intermedium, M kansasii, M komossense, M. kubicae, M lentiflavum, M. leprae, M. lepraemurium, M luteum, M. madagascariense, M mageritense, M malmoense, M marinum, M. microti, M moriokaense, M. mucogenicum, M. murale, M. neoaurum, M. nonchromogenicum, M. novocastrense, M. obuense, M. parqfortuitum, M. paratuberculosis, M peregrinum, M phage, M. phlei, M. porcinum, M poriferae, M. pulveris, M. rhodesiae, M scrofulaceum, M. senegalense, M. septicum, M. shimoidei, M. simiae, M smegmatis, M. sphagni, M. szulgai, M. terrae, M thermoresistibile, M. tokaiense, M. triplex, M triviale, M. tuberculosis, M. tusciae, M ulcerans, M. vaccae, M. wolinskyi, M xenopi, Neisseria animalis, N. canis, N. cinerea, N. denitrificans, N. dentiae, N. elongata, N. flava, N. flavescens, N. gonorrhoeae, N. iguanae, N. lactamica, N. macacae, N. meningitidis, N. mucosa, N. ovis, N. perflava, N. phalyngis var. flava, N. polysaccharea, N. sicca, N. subflava, N. weaveri, Pseudomonas aeruginosa, P. alcaligenes, P. chlororaphis, P. fluorescens, P. luteola, P. mendocina, P. monteilii, P. olyzihabitans, P. pertocinogena, P. pseudalcaligenes, P. putida, P. stutzeri, Salmonella bacteriophage, S. bongori, S. choleraesuis, S. enterica, S. enteritidis, S. paratyphi, S. typhi, S. typhimurium, S. typhimurium, S. typhimurium, S. typhimurium bacteriophage, Shigella boydii, S. dysenteriae, S. flexneri, S. sonnei, Staphylococcus arlettae, S. aureus, S. auricularis, S. bacteriophage, S. capitis, S. caprae, S. carnosus, S. caseolyticus, S. chromogenes, S. cohnii, S. delphini, S. epidermidis, S. equorum, S. felis, S. fleurettii, S. gallinarum, S. haemolyticus, S. hominis, S. hyicus, S. intermedius, S. kloosii, S. lentus, S. lugdunensis, S. lutrae, S. muscae, S. mutans, S. pasteuri, S. phage, S. piscifermentans, S. pulvereri, S. saccharolyticus, S. saprophyticus, S. schleiferi, S. sciuri, S. simulans, S. succinus, S. vitulinus, S. warneri, S. xylosus, Ureaplasma urealyticum, Yersinia aldovae, Y bercovieri, Y enterocolitica, Y frederiksenii, Y intermedia, Y kristensenii, Y mollaretii, Y pestis, Y philomiragia, Y pseudotuberculosis, Y rohdei, and Y ruckeri.

Ribosomes from obligate intracellular bacteria, such as: Anaplasma bovis, A. caudatum, A. centrale, A. marginate A. ovis, A. phagocytophila, A. platys, Bartonella bacilliform is, B. clarridgeiae, B. elizabethae, B. henselae, B. henselae phage, B. quintana, B. taylorii, B. vinsonii, Borrelia afzelii, B. andersonii, B. anserina, B. bissettii, B. burgdorferi, B. crocidurae, B. garinii, B. hermsii, B. japonica, B. miyamotoi, B. parkeri, B. recurrentis, B. turdi, B. turicatae, B. valaisiana, Brucella abortus, B. melitensis, Chlamydia pneumoniae, C. psittaci, C. trachomatis, Cowdria ruminantium, Coxiella burnetii, Ehrlichia canis, E. chaffeensis, E. equi, E. ewingii, E. muris, E. phagocytophila, E. platys, E. risticii, E. ruminantium, E. sennetsu, Haemobartonella canis, H. felis, H. muris, Mycoplasma arthriditis, M buccale, M. faucium, M. fermentans, M genitalium, M hominis, M. laidlawii, M. lipophilum, M orale, M. penetrans, M. pirum, M. pneumoniae, M. salivarium, M. spermatophilum, Rickettsia australis, R. conorii, R. felis, R. helvetica, R. japonica, R. massiliae, R. montanensis, R. peacockii, R. prowazekii, R. rhipicephali, R. rickettsii, R. sibirica, and R. typhi.

Ribosomes from facultative intracellular fungi, such as: Candida Candida aaseri, C. acidothermophilum, C. acutus, C. albicans, C. anatomiae, C. apis, C. apis var. galacta, C. atlantica, C. atmospherica, C. auringiensis, C. bertae, C. berthtae var. chiloensis, C. berthetii, C. blankii, C. boidinii, C. boleticola, C. bombi, C. bombicola, C. buinensis, C. butyri, C. cacaoi, C. cantarellii, C. cariosilignicola, C. castellii, C. castrensis, C. catenulata, C. chilensis, C. chiropterorum, C. coipomensis, C. dendronema, C. deserticola, C. diddensiae, C. diversa, C. entomaea, C. entomophila, C. ergatensis, C. ernobii, C. ethanolica, C. ethanothermophilum, C. famata, C. fluviotilis, C. fragariorum, C. fragicola, C. friedrichii, C. fructus, C. geochares, C. glabrata, C. glaebosa, C. gropengiesseri, C. guilliermondii, C. guilliermondii var. galactosa, C. guilliermondii var. soya, C. haemulonii, C. halophila/C. versatilis, C. holmii, C. humilis, C. hydrocarbofumarica, C. inconspicua, C. insectalens, C. insectamans, C. intermedia, C. javanica, C. kefyr, C. krissii, C. krusei, C. krusoides, C. lambica, C. lusitaniae, C. magnoliae, C. maltosa, C. mamillae, C. maxis, C. maritima, C. melibiosica, C. melinii, C. methylica, C. milleri, C. mogii, C. molischiana, C. montana, C. multis-gemmis, C. musae, C. naeodendra, C. nemodendra, C. nitratophila, C. norvegensis, C. norvegica, C. oleophila, C. oregonensis, C. osornensis, C. paludigena, C. parapsilosis, C. pararugosa, C. periphelosum, C. petrohuensis, C. petrophilum, C. philyla, C. pignaliae, C. pintolopesii var. pintolopesii, C. pintolopesii var. slooffiae, C. pinus, C. polymorpha, C. populi, C. pseudointermedia, C quercitrasa, C. railenensis, C. rhagii, C. rugopelliculosa, C. rugosa, C. sake, C. salmanticensis, C. savonica, C. sequanensis, C. shehatae, C. silvae, C. silvicultrix, C. solani, C. sonorensis, C. sorbophila, C. spandovensis, C. sphaerica, C. stellata, C. succiphila, C. tenuis, C. terebra, C. tropicalis, C. utilis, C. valida, C. vanderwaltii, C. vartiovaarai, C. veronae, C. vini, C. wickerhamii, C. xestobii, C. zeylanoides, and Histoplasma capsulatum.

Ribosomes from obligate intracellular protozoans, such as: Brachiola vesicularum, B. connori, Encephalitozoon cuniculi, E. hellem, E. intestinalis, Enterocytozoon bieneusi, Leishmania aethiopica, L. amazonensis, L. braziliensis, L. chagasi, L. donovani, L. donovani chagasi, L. donovani donovani, L. donovani infantum, L. enriettii, L. guyanensis, L. infantum, L. major, L. mexicana, L. panamensis, L. peruviana, L. pifanoi, L. tarentolae, L. tropica, Microsporidium ceylonensis, M. africanum, Nosema connori, N. ocularum, N. algerae, Plasmodium berghei, P. brasilianum, P. chabaudi, P. chabaudi adami, P. chabaudi chabaudi, P. cynomolgi, P. falciparum, P. fragile, P. gallinaceum, P. knowlesi, P. lophurae, P. malariae, P. ovale, P. reichenowi, P. simiovale, P. simium, P. vinckeipetteri, P. vinckei vinckei, P. vivax, P. yoelii, P. yoelii nigeriensis, P. yoelii yoelii, Pleistophora anguillarum, P. hippoglossoideos, P. mirandellae, P. ovariae, P. typicalis, Septata intestinalis, Toxoplasma gondii, Trachipleistophora hominis, T. anthropophthera, Vittaforma corneae, Trypanosoma avium, T. brucei, T brucei brucei, T brucei gambiense, T brucei rhodesiense, T cobitis, T. congolense, T cruzi, T. cyclops, T. equiperdum, T. evansi, T dionisii, T godfreyi, T grayi, T lewisi, T. mega, T microti, T pestanai, T. rangeli, T rotatorium, T simiae, T. theileri, T varani, T vespertilionis, and T. vivax.

The ribosomes from the pathogen may be bound to the support, i.e., immobilized, via a binding agent Immobilizing complexes such as ribosomes to a support is well within the skill of one in the art. In certain embodiments, the binding agent is capable of specifically binding a ribosome, e.g. is specific for a ribosome from a particular pathogen. For example, the binding agent may be a polyclonal or monoclonal antibody or an antibody fragment, e.g. specific for the ribosome to be captured. For example, the antibody or antibody fragment may be specific for a ribosomal protein comprising the ribosome. In other embodiments, the binding agent is a ribosomal binding protein specific for the ribosome to be captured. In other embodiments, the binding agent is capable of binding ribosomal RNA, and may be a nucleic acid, i.e., a nucleic acid complementary to and specific for the ribosomal RNA of the ribosome to be captured. In still other embodiments, the binding agent is a protein that binds ribosomal RNA, e.g., an RNA binding protein.

The support may be any suitable material for immobilizing ribosomes via any of the above-described binding agents. In certain embodiments, the support is a porous material, e.g. nitrocellulose. In other embodiments, the support is a particle, e.g., a magnetic particle. In certain embodiments, the support is comprised of a plurality of particles.

In certain embodiments, the support is part of a test strip or test chamber. In some embodiments, the support is a test strip. In other embodiments, the support is part of a lateral flow device. In still other embodiments, the support is resin or other material suitable for packing in a column. In still other embodiments, the support is a plate with wells or a test tube.

The support may be washed with any buffer appropriate to maintaining the biological function of the ribosomes yet sufficient to remove unbound ribosomes and other material. The wash buffer must contain suitable inhibitors of ribosomal activity in order to ensure the residual ribosomes do not translate mRNA. Alternatively, or in addition to adding inhibitors, the wash buffer may be autoclaved to destroy ribosomal activity. Appropriate pH, glycerol or other stabilizing molecules such as ligands, polyethylene glycol, etc., and the presence or absence of reducing agent, chelating agent, cofactors, detergents, protease inhibitors, ribosomal activity inhibitors are accordingly all important considerations. The support may be washed with anywhere from about 5 to 20 volumes of each wash buffer to eliminate unbound ribosomes from the support, e.g., in embodiments wherein the support is or is comprised within a column, lateral flow, or other format wherein the wash may flow over the support. In certain embodiments wherein the support is a particle, the washing may comprise moving the particles from one liquid, e.g., the sample, to another liquid, e.g., the wash. In certain embodiments, the methods described above may be modified to detect viruses. In the case of RNA viruses, a sample potentially comprising an RNA virus may be flowed over a surface comprising antibodies against the virus and ribosomes. As the sample is flowed over the surface, viruses, if present may be captured by the antibodies. After a wash step as described above to remove unbound materials, the viruses may be lysed by flowing a lysis solution over the surface comprising the bound viruses. The lysis releases the RNA, which is available for translation by the ribosomes as follows.

After washing, the support is incubated in the presence of a solution comprising mRNA and amino acids under conditions suitable to allow ribosomes bound to the binding agent to translate the mRNA in the solution into a polypeptide. In certain embodiments, constant temperature is maintained during the incubation.

The solution may include one or more energy sources providing chemical energy for protein synthesis. Further, the solution includes at least one nucleic acid template, for example, an mRNA. The solution may include enzymes, translation factors or co-factors. In certain embodiments, the solution may include E. coli rare t-RNAs selected from tRNAs for amino acids arginine, proline, glycine, leucine or isoleucine. Further, the solution may include lipids, cholesterol, or membranes.

The solution may also include an inhibitor of an enzyme that degrades the template or other enzymes necessary for the translation reaction, such as phosphatases, proteases, nucleases, deoxyribonucleases, or ribonucleases. Further, it may include an enzyme to catalyze hydrolysis or formation of phosphodiester bonds.

Still further, the solution may include at least one molecular chaperone or a foldase. The molecular chaperone or foldase includes, but is not limited to, GroEL/ES, GroEL, GroES, TF, DnaK, DnaJ, GrpE, ClpB, FkpA, Skp, DsbA, DsbC, peptidyl prolyl cis/trans isomerase (PPI), chaperonin 60, chaperonin 10, TCP1, TF55, heat shock protein 60, Cpn60, heat shock protein 10, Cpn10, Lim protein, or signal recognition particle.

The amount of protein produced in a translation reaction can be measured by various means, such as specific enzymatic activity, UV or visible light absorption or fluorescence. Products of protein synthesis may also be detected by using antibody based assays. Another method to quantitate the amount of protein produced in a coupled in vitro transcription translation reactions is to perform the reaction using a known quantity of radiolabeled amino acid such as ³⁵S-methionine or ³H-leucine and subsequently measuring the amount of radiolabeled amino acid incorporated into the newly synthesized protein. Incorporation assays will measure the amount of radiolabeled amino acids in all proteins produced in an in vitro translation reaction including truncated protein products.

Other methods for detecting nascent proteins are described in U.S. Published Patent Application 20050032078.

For use in the diagnostic and other applications suggested above, kits and devices for the practice of the above-described methods are also provided. Devices for practice of the methods include lateral flow devices (wherein the reagents employed in the reaction may be dried onto the chromatographic support contained within the device), a test strip, or other support for practice of the methods. A kit may include a nucleic acid template, a binding agent specific for ribosomes of a pathogen of interest, a support, and wash and incubation buffers. Such kits and devices can contain any number or combination of reagents or components. The kits can comprise one or more of the above components in any number of separate containers, tubes, vials and the like or such components can be combined in various combinations in such containers. Kit components may be packaged for either manual or partially or wholly automated practice of the foregoing methods. Further, instructions for the use of a device or kit may be included with the device or kit. Such kits and devices may have a variety of uses, including, for example, diagnosis, therapy, and other applications

### EXAMPLE

The invention, having been generally described, may be more readily understood by reference to the following examples, which are included merely for purposes of illustration of certain aspects and embodiments of the present invention, and are not intended to limit the invention in any way. All headings are for the convenience of the reader and should not be used to limit the meaning of the text that follows the heading, unless so specified.

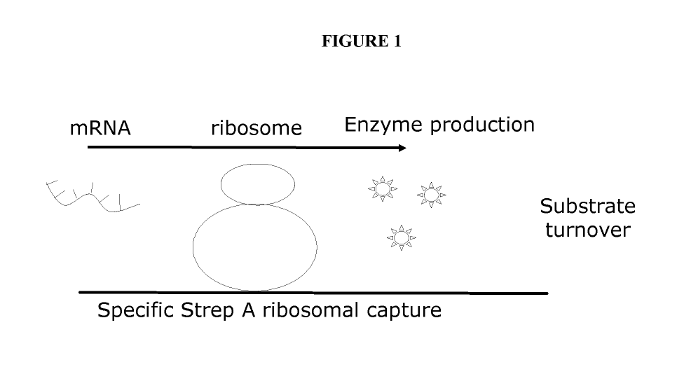

FIG. 1 depicts the use of ribosomal amplification to detect Streptococcus A bacteria. Briefly, antibody to a specific ribosomal protein of Streptococcus A is bound on a support. The sample to be tested is extracted and the bacteria contained within the sample lysed to allow ribosomes to be released (approximately 20,000 ribosomes per bacterial cell). The sample is then flowed over the support to allow the released ribosomes to bind the antibody. The sample is washed to remove unbound ribosomes and other materials from the support, and a generic solution of mRNA encoding an enzyme, amino acids, and substrate is flowed over the support. The ribosomes bound to the antibody should translate mRNA into the enzyme continuously at a consistent temperature compared to PCR. The enzyme upon translation breaks the substrate down, which change may be detected optically.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

We claim:

1. A method, comprising:
(a) contacting a support comprising a binding agent capable of binding a ribosome with a sample comprising ribosomes;
(b) washing the support after contact with the sample;
(c) incubating the support in the presence of a solution comprising mRNA and amino acids under conditions suitable to allow ribosomes bound to the binding agent to translate the mRNA in the solution into a polypeptide; and
(d) determining the presence of the polypeptide.

2. The method of claim 1, wherein the ribosomes are specific to a pathogen and the method further comprises determining the presence of the pathogen in the sample based on the presence of the polypeptide.

3. The method of claim 2, wherein the pathogen is a bacterium.

4. The method of claim 1, wherein the binding agent is capable of specifically binding a ribosome.

5. The method of claim 3, wherein the ribosomes are bacterial ribosomes and the method further comprises releasing the ribosomes from at least one bacterium.

6. The method of claim 1, wherein the support is a porous material.

7. The method of claim 3, wherein the porous material is nitrocellulose.

8. The method of claim 1, wherein the binding agent is an antibody.

9. The method of claim 1, wherein the binding agent is an agent capable of binding ribosomal RNA.

10. The method of claim 9, wherein the agent capable of binding ribosomal RNA is a nucleic acid.

11. The method of claim 1, wherein the solution comprising mRNA and amino acids further comprises a substrate.

12. The method of claim 11, wherein the polypeptide is an enzyme.

13. The method of claim 12, wherein determining the polypeptide comprises determining the presence of a reaction product resulting from reaction of the enzyme with a substrate for the enzyme.

14. The method of claim 13, wherein determining the polypeptide comprises optically detecting the presence of the reaction product.

15. A method, comprising:
(a) capturing ribosomes on a support using a binding agent capable of binding the ribosomes;
(b) incubating the captured ribosomes with a solution comprising mRNA and amino acids; and
(c) detecting the polypeptide produced by the incubated, captured ribosomes.

16. A device, comprising a support comprising a binding agent capable of binding a ribosome.

17. The device of claim 16, wherein the support is a porous material.

18. The device of claim 17, wherein the porous material is nitrocellulose.

19. The device of claim 16, wherein the binding agent is an antibody.

20. The device of claim 16, wherein the binding agent is an agent capable of binding ribosomal RNA.

21. The device of claim 20, wherein the agent capable of binding ribosomal RNA is a nucleic acid.
